(19) United States
(12) Patent Application Publication
(10) Pub. No.: US 2018/0303812 A1
(43) Pub. Date: Oct. 25, 2018

(54) PHARMACEUTICAL COMPOSITION FOR USE IN TREATING AML AND METHOD OF TREATING AML IN A SUBJECT IN NEED THEREOF

(71) Applicants: DAIICHI SANKYO COMPANY, LIMITED, Tokyo (JP); BOARD OF REGENTS, THE UNIVERSITY OF TEXAS SYSTEM, Austin, TX (US)

(72) Inventors: Kenji NAKAMARU, Tokyo (JP); Koichi TAZAKI, Tokyo (JP); Takahiko SEKI, Tokyo (JP); Ngai-chiu Archie TSE, Long Island City, NY (US); Michael ANDREEFF, Houston, TX (US); Jo ISHIZAWA, Houston, TX (US)

(73) Assignees: DAIICHI SANKYO COMPANY, LIMITED, Tokyo (JP); BOARD OF REGENTS, THE UNIVERSITY OF TEXAS SYSTEM, Austin, TX (US)

(21) Appl. No.: 15/769,308

(22) PCT Filed: Oct. 21, 2016
(86) PCT No.: PCT/JP2016/081974

§ 371 Date: Apr. 18, 2018

Related U.S. Application Data
(60) Provisional application No. 62/245,667, filed on Oct. 23, 2015.

(51) Int. Cl. A61K 31/4439; A61P 35/02

(52) U.S. Cl. CPC A61K 31/4439; G01N 33/57484; A61P 35/02

(57) ABSTRACT

The present invention provides a pharmaceutical composition for use in treating acute myeloid leukemia (AML) and a method of treating AML in a patient in need thereof. The present invention also provides a method for predicting the sensitivity to the treatment in the patient using gene expression signature.

### FIELD OF THE INVENTION

The present invention relates to a pharmaceutical composition for use in treating acute myeloid leukemia (AML) and a method of treating AML in a patient in need thereof. The present invention also relates to a method for predicting the sensitivity to the treatment in the patient using gene expression signature.

### BACKGROUND OF THE INVENTION

MDM2, located on Chromosome 12 q13-15, is a negative regulator of the p53 tumor suppressor protein. The 90 kDa MDM2 protein contains a p53 binding domain at its N-terminus and a RING (really interesting gene) domain at its C-terminus, which functions as an E3 ligase that ubiquitinates p53. The activation of wild-type p53 by cell stimuli and stresses results in the binding of MDM2 to p53 at the N-terminus to inhibit the transcriptional activation of p53 and promote the degradation of p53 via the ubiquitin-proteasome pathway. Thus, MDM2 can interfere with p53-mediated apoptosis and arrest of cancer cell proliferation, attributing a significant oncogenic activity to MDM2 in cancer cells. In some cases, MDM2 can cause carcinogenesis independent of the p53 pathway, for example, in cells which possess an alternative splice form of MDM2 (H. A. Steinman et al., 2004, J. Biol. Chem., 279(6):4877-4886). Therefore, several MDM2 inhibitors have been developed to treat cancers, including (3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrro lidine-3′,3″-indole]-5′-carboxamide (WO2012/121361, US Patent Application Publication No. 2012/0264738A and WO2015/108175). Overexpression of MDM2 has been reported to correlate positively with poor prognosis in individuals having sarcoma, glioma and acute lymphoblastic leukemia (ALL).

Acute myeloid leukemia (AML) is a hematological malignancy derived from stem or myeloid progenitor cells in the bone marrow, which is also called acute myeloid leukemia or acute nonlymphocytic leukemia ANLL). The symptoms of AML include fatigue, bleeding disorder and increased risk in infections. In a patient suffering from AML, the normal bone marrow is replaced with leukemic blasts. Many pharmaceuticals have been developed, some of which are MDM2 inhibitors such as Nutlin-3 (Kojima et al. 2005; Blood 106(9):3150-3159; Secchiero et al., 2007, Neoplasia, 9(10): 853-861), and MI219 (Long et al., 2010, Blood, 116: 71-80). The first clinical trial in AML with the MDM2 inhibitor RG7112 was reported to induce clinical responses including complete remissions (Andreeff, M. et al., Clin. Cancer Res. 2015, epubl.)

### SUMMARY OF THE INVENTION

The present invention provides a pharmaceutical composition for use in treating acute myeloid leukemia (AML) and a method of treating AML in a patient in need thereof. The present invention also provides a method for predicting the sensitivity to the treatment in the patient using gene expression signature.

The inventors have discovered that AML can be therapeutically treated with (3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrro lidine-3′,3″-indole]-5′-carboxamide or a salt thereof, which is a highly potent dispiropyrrolidine-based MDM2 inhibitor represented by the formula (I) below. The inventors have also discovered that the sensitivity to the aforementioned compound in an AML subject is predictable.

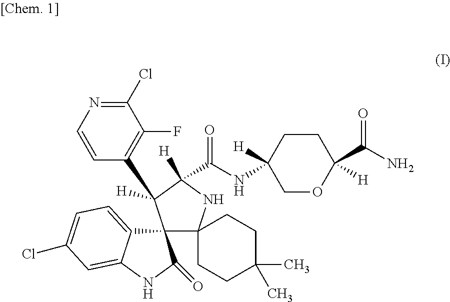 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

The present invention provides:

(1) A pharmaceutical composition for use in treating acute myeloid leukemia (AML) in a patient in need thereof, comprising a therapeutically effective amount of (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrol idine-3′,3″-indole]-5′-carboxamide or a salt thereof and a pharmaceutically acceptable carrier.
(2) The pharmaceutical composition according to above (1), wherein the salt is p-toluenesulfonic acid salt monohydrate.
(3) The pharmaceutical composition according to above (1) or (2), wherein the patient has been predicted as being sensitive to the treatment by measuring the expression level of at least one gene or all gene selected from a group of the 177 genes listed in FIG. 1 in a sample obtained from the patient.
(4) The pharmaceutical composition according to above (3), wherein the patient has been predicted as being sensitive to the treatment by measuring the expression levels of the 177 genes listed in FIG. 1 in a sample obtained from the patient.
(5) The pharmaceutical composition according to above (3), wherein the patient has been predicted as being sensitive to the treatment by measuring the expression levels of the 175 genes which are listed in FIG. 1 except for EDA2R and SPATA18 in a sample obtained from the patient.
(6) The pharmaceutical composition according to above (3), wherein the patient has been predicted as being sensitive to the treatment by measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in a sample obtained from the patient.
(7) The pharmaceutical composition according to above (3), wherein the patient has been predicted as being sensitive to the treatment by measuring the expression levels of at least one gene or all genes selected from the group of the genes below: RPS27L, FDXR, CDKN1A and AEN in a sample obtained from the patient.
(8) The pharmaceutical composition according to above (3), wherein the patient has been predicted as being sensitive to the treatment by measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in a sample obtained from the patient.
(9) The pharmaceutical composition according to above (3), wherein the patient has been predicted as being sensitive to the treatment by measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in a sample obtained from the patient.
(10) The pharmaceutical composition according to above (3), (4), (5), (6), (7), (8), or (9), wherein the patient has a wild type TP53 gene in the genome of AML cells to be treated.
(11) A method of treating acute myeloid leukemia (AML) in a patient in need thereof, comprising administering to the patient a therapeutically effective amount of (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrol idine-3′,3″-indole]-5′-carboxamide or a salt thereof.
(12) The method according to above (11), wherein the salt is p-toluenesulfonic acid salt monohydrate.
(13) The method according to above (11) or (12), wherein the patient has been predicted as being sensitive to the treatment by measuring the expression level of at least one gene or all genes selected from a group of the 177 genes listed in FIG. 1 in a sample obtained from the patient.
(14) The method according to above (13), wherein the patient has been predicted as being sensitive to the treatment by measuring the expression levels of the 177 genes listed in FIG. 1 in a sample obtained from the patient.
(15) The method according to above (13), wherein the patient has been predicted as being sensitive to the treatment by measuring the expression levels of the 175 genes which are listed in FIG. 1 except for EDA2R and SPATA18 in a sample obtained from the patient.
(16) The method according to above (13), wherein the patient has been predicted as being sensitive to the treatment by measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in a sample obtained from the patient.
(17) The method according to above (13), wherein the patient has been predicted as being sensitive to the treatment by measuring the expression levels of at least one gene or all genes selected from the group of the genes below: RPS27L, FDXR, CDKN1A and AEN in a sample obtained from the patient.
(18) The method according to above (13), wherein the patient has been predicted as being sensitive to the treatment by measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in a sample obtained from the patient.
(19) The method according to above (13), wherein the patient has been predicted as being sensitive to the treatment by measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in a sample obtained from the patient.
(20) The method according to above (13), (14), (15), (16), (17), (18), or (19), wherein the patient has a wild type TP53 gene in the genome of AML cells to be treated.
(21) A method of predicting sensitivity to MDM2i treatment in a patient suffering from AML, comprising measuring the expression levels of at least one, at least two, at least three, at least four or all of the 177 signature genes shown in FIG. 1.
(22) The method according to above (21), comprising measuring the expression levels of at least one, at least two, at least three, at least four or all of the 175 signature genes which are the genes presented in FIG. 1 except for EDA2R and SPATA18.
(23) The method according to above (21), comprising measuring the expression levels of at least one, at least two, at least three, at least four or all of the forty signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.
(24) The method according to above (21), comprising measuring the expression levels of RPS27L, FDXR, CDKN1A and AEN.
(25) The method according to any one of above (21) to (24), further comprising determining whether or not the AML has a wild-type TP53 gene in its genome.
(26) A method of predicting sensitivity to MDM2i treatment in a patient suffering from AML comprising,

determining whether or not the AML has mutant TP53 gene in its genome,
when the AML has mutant TP53 gene, then the patient is predicted as resistant, and

when the AML has wild-type TP53 gene, subsequently measuring the expression levels of at least one, at least two, at least three, at least four or all of the 177 signature genes shown in FIG. 1 in the AML,
when the AML has a low signature score compared to a predetermined cutoff value, then the patient is predicted as resistant, and
when the AML has a high signature score compared to the predetermined cutoff value, then the patient is predicted as sensitive.
   (27) The method according to above (26), wherein the step of measuring is measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in the AML.
   (28) The method according to above (26) or (27), wherein the step of measuring is measuring the expression levels of at least one gene or all genes selected from the group of the genes below: RPS27L, FDXR, CDKN1A and AEN in the AML.
   (29) The method according to any one of above (26) to (28), wherein the step of measuring is measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in the AML.
   (30) The method according to any one of above (26) to (29), wherein the step of measuring is measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in the AML.

### BRIEF DESCRIPTION OF THE DRAWINGS

FIG. 1 presents in tabular format 177 gene signature biomarkers that are differentially expressed in cancer or tumor samples or cells that are sensitive to the MDM2i, as described herein.

FIGS. 2A and 2B present the representative results of detection of % live cell in the sensitive samples after the 48-hour exposure to Compound 2. The results in Sample Nos. 2 and 15 were shown in FIGS. 2A and 2B, respectively.

FIG. 3 presents the relationship between the measured sensitivity and the predicted sensitivity (sensitivity score) of each TP53 wild type sample to the compound of formula (I) (the upper panel) in the prediction using the 175 gene signature, and the Receiver Operating Characteristic (ROC) curve in the prediction (the lower panel). The vertical line in the upper panel indicates the cutoff value in the prediction.

FIG. 4 presents the relationship between the measured sensitivity and the predicted sensitivity (sensitivity score) of each TP53 wild type sample to the compound of formula (I) (the upper panel) in the prediction using the 40 gene signature, and the Receiver Operating Characteristic (ROC) curve in the prediction (the lower panel). The vertical line in the upper panel indicates the cutoff value in the prediction.

FIG. 5 presents the relationship between the measured sensitivity and the predicted sensitivity (sensitivity score) of each of all samples to the compound of formula (I) (the upper panel) in the prediction using the 175 gene signature, and the Receiver Operating Characteristic (ROC) curve in the prediction (the lower panel). The vertical line in the upper panel indicates the cutoff value in the prediction.

FIG. 6 presents the relationship between the measured sensitivity and the predicted sensitivity (sensitivity score) of each of all samples to the compound of formula (I) (the upper panel) in the prediction using the 40 gene signature, and the Receiver Operating Characteristic (ROC) curve in the prediction (the lower panel). The vertical line in the upper panel indicates the cutoff value in the prediction.

FIG. 7 presents a prediction scheme for MDM2i sensitivity in a preferred embodiment of the invention.

### DETAILED DESCRIPTION OF THE INVENTION

The term “comprise” as used herein is intended to be an open-ended, inclusive and does not exclude additional, unrecited features, and then encompasses the closed term “consist of” or “essentially consist of”.

The term “patient” refers to a mammal, especially a human, suffering from AML. The patient may be a patient who has been or was previously treated by other therapy. The patient may also be a patient with newly diagnosed, relapsed or refractory AML. The patient may also be a patient suffering from myelodysplastic syndromes, or a patient with newly diagnosed or relapsed myelodysplastic syndromes.

The term “treating” in a general sense refers to achieving or obtaining a desired physiologic and/or pharmacologic effect, whether prophylactic, therapeutic, or both. Treatment of a patient in need thereof typically involves the use or administration of an effective amount or a therapeutically effective amount of the compound. Effective amount refers to the quantity (amount) of the compound that induces a desired response in a patient upon administration or delivery to the patient.

The term “MDM2” refers to an E3 ubiquitin ligase which can interact with p53 and cause p53 degradation. MDM2 includes, but is not limited to, mouse MDM2 and the human ortholog of MDM2 (also called “Human Double Minute 2” or “HDM2”). The term “MDM2 inhibitor” refers to an inhibitor inhibiting MDM2 functions or activities on p53 degradation.

The term “MDM2i” encompasses a number of low molecular weight MDM2 inhibitors.

The term “Compound 1” as used herein refers to (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrol idine-3′,3″-indole]-5′-carboxamide as represented by the formula (I) or pharmaceutically acceptable salt thereof. The term “Compound 2” as used herein refers to the p-toluenesulfonic acid salt monohydrate of Compound 1. These compounds can be prepared by those skilled in the art according to WO2012/121361, which is herein incorporated by reference in its entirety. The term “Compound 1 treatment” as used herein refers to the treatment of an AML subject with Compound 1, preferably with compound 2.

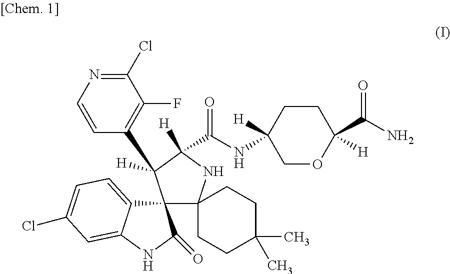 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

The term “array” as used herein refers to an arrangement, typically an ordered arrangement, of biological molecules, e.g., nucleic acids, polypeptides, peptides, biological samples, placed in discrete, assigned and addressable locations on or in a surface, matrix, or substrate. Microarrays are miniaturized versions of arrays that are typically evaluated or analyzed microscopically. Nucleic acid, e.g., RNA or DNA, arrays are arrangements of nucleic acids (such as probes) in assigned and addressable locations on a solid surface or matrix. Nucleic acid arrays encompass cDNA arrays and oligonucleotide arrays and microarrays; they may be referred to as biochips, or DNA/cDNA chips. Microarrays, as well as their construction, reagent components and use are known by those having skill in the pertinent art. By way of example, microarray technology useful for determining and measuring gene expression status is provided in US 2011/0015869.

The term “biomarker” generally refers to a gene, an expressed sequence tag (EST) derived from the gene, a set of genes, or a set of proteins or peptides whose expression levels change under certain conditions, or differ in certain cellular contexts, such as in cells sensitive to a certain treatment as opposed to those that are insensitive to the treatment. In general, when the expression levels of the genes or gene sets correspond to a certain condition, the gene(s) serve(s) as one or more biomarkers for that condition. Biomarkers can be differentially expressed among individuals, (e.g., those with a cancer or tumor type) according to prognosis and disease state; thus, biomarkers may be predictive of different survival outcomes, as well as of the benefit drug susceptibility and sensitivity.

The term “gene” as used herein refers to a DNA sequence which is expressed in a subject as an RNA transcript; a gene can be a full-length gene (protein encoding or non-encoding).

The terms “gene signature”, “gene expression signature” and “gene sensitivity signature” are used interchangeably herein as they refer to the expression, such as differential expression, or the expression patterns, of genes predictive of cellular response in AML sensitive to the treatment with Compound 1 in accordance with the invention. For example, in an embodiment, AML samples showing sensitivity to the treatment with Compound 1 have increased or elevated levels of expression of genes contained in the gene signatures of the invention compared with a control.

As used in accordance with the present invention, “gene expression” means the process of converting genetic information encoded in a gene into RNA (e.g., mRNA, rRNA, tRNA, or snRNA) through transcription of the gene (e.g., as mediated by the enzymatic action of an RNA polymerase), and for protein-encoding genes, into protein through “translation” of mRNA.

Alterations in gene expression, such as differential expression, can include, without limitation, overexpression, increased expression, underexpression, or suppressed expression, as compared to a control, such as non-cancer cells or in relation to normalized expression levels. Alterations in the expression of a nucleic acid molecule may be associated with, and in some instances cause, a change in expression of the corresponding protein. Illustratively, gene expression can be measured to determine differential expression of genes in the gene signatures indicative of the sensitivity of a patient's AML sample to the treatment with Compound 1 in order to predict the patient's likelihood of responding to the treatment for the purpose of administering Compound 1 to the patient, and/or personalizing an effective treatment with Compound 1, and/or predicting the patient's survival time.

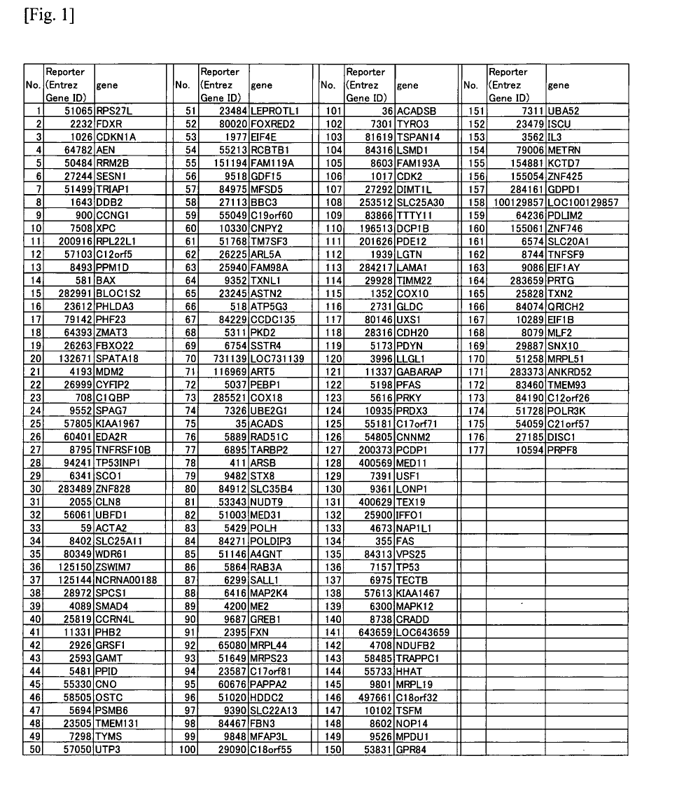

An increase in expression, which may also be referred to as upregulated or activated expression, used in reference to a gene or nucleic acid molecule, refers to any process that causes or results in increased or elevated production of a gene product, such as all types of RNA, or protein. Increased or elevated gene expression includes any process that increases the transcription of a gene or the translation of mRNA into protein. Increased (or upregulated) gene expression can include any detectable or measurable increase in the production of a gene product. Illustratively, the production of a gene product, (such as at least three, at least four, or all, of the genes of FIG. 1; or at least one, at least two, at least three, at least four, or all, of the gene genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; at least one, at least two, at least three, at least four, or all, of the genes consisting of MDM2, CDKN1A, ZMAT3, DDB2, FDXR, RPS27L, BAX, RRM2B, SESN1, CCNG1, XPC, TNFRSF10B and AEN; at least one, at least two, at least three, at least four, or all, of the genes consisting of BAX, RPS27L, EDA2R, XPC, DDB2, FDXR, MDM2, CDKN1A, TRIAP1, BBC3, CCNG1, TNFRSF10B, and CDKN2A; or at least one, at least two, at least three, or all, of the gene signature genes consisting of RPS27L, FDXR, CDKN1A and AEN), is increased by a measurable, relative amount, for example, and without limitation, an increase of at least 1.5-fold, at least 2-fold, at least 3-fold, at least 4-fold, at least 5-fold, or at least 6-10 fold, as compared to a control. The control may be the amount of gene expression in a biological sample, such as a normal cell, or a reference value, or a normalized value of cellular gene expression. In an example, a control is the relative amount of gene expression in a biopsy of the same tissue type from a subject who does not have AML, as does the subject in question (who is undergoing testing). In another example, a control is the relative amount of gene expression in a tissue biopsy from non-tumor tissue of the same tissue type (such as peripheral blood and bone marrow) as that of the tumor, taken from the subject having the tumor and undergoing testing.

In some cases, expression levels of the disclosed genes (such as expression of at least one, at least two, at least three, at least four, at least five, at least six, at least ten, or all, of the genes listed in the gene signatures of FIG. 1; at least one, at least two, at least three, at least four, or all, of the genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; at least one, at least two, at least three, at least four, or all, of the genes consisting of MDM2, CDKN1A, ZMAT3, DDB2, FDXR, RPS27L, BAX, RRM2B, SESN1, CCNG1, XPC, TNFRSF10B and AEN; at least one, at least two, at least three, at least four, or all, of the genes consisting of BAX, RPS27L, EDA2R, XPC, DDB2, FDXR, MDM2, CDKN1A, TRIAP1, BBC3, CCNG1, TNFRSF10B, and CDKN2A; or at least one, at least two, at least three, or all, of the genes consisting of RPS27L, FDXR, CDKN1A and AEN) are normalized relative to the expression levels of one or more housekeeping genes, e.g., in the same or different cancer or neoplasm sample. An aggregate value is obtained in some cases by calculating the level of expression of each of the genes (e.g., each of the genes in a gene signature) and using a positive or negative weighting for each gene depending on whether the gene is positively or negatively regulated by a condition (e.g., sensitivity to Compound 1 treatment or a survival risk score). In some cases, the normalized expression of the gene or the gene signature, or an aggregate value, is determined to be increased or decreased relative to the median normalized expression of the gene or gene signature, or to an aggregate value, for a set of cancers or cancer types. In some cases, the median normalized expression or aggregate value is obtained from publicly-available microarray datasets, such as leukemia, lymphoma, melanoma, or myeloma cancer microarray datasets. In an example, a median normalized expression or aggregate value for expression genes of the gene signature is determined using microarray datasets.

In some cases, a score (sensitivity score) is calculated from the normalized expression level measurements. The score can be utilized to provide cutoff points or values to identify various parameters, such as AML as being sensitive, or less likely to be sensitive, to Compound 1 and/or low, medium, or high sensitivity of a patient with AML to Compound 1 treatment or therapy. In some cases, the cutoff points are often determined using training and validation datasets. By way of example, a supervised approach can be utilized to establish the cutoff that distinguishes those who will be sensitive (responders) from those who will not respond to Compound 1 treatment, for example, by comparing gene signature expression in responders and non-responders. In another example, an unsupervised approach can be utilized to determine empirically a cutoff level (for example, top 50% versus bottom 50%, top quartile versus bottom quartile, or top tercile versus bottom tercile) that is predictive of an outcome, i.e., sensitivity to Compound 1 treatment. The cutoff determined in the training set can be tested in one or more independent validation datasets.

The term “diagnose” refers to the recognition or identification of a disease or condition by signs or symptoms, frequently involving the use of external tests, evaluations and analyses. A diagnosis of the disease or condition results from the entirety of the procedures involved in making and drawing a conclusion to identify the disease or condition. According to the invention, the sensitivity of a patient to Compound 1, as well as the likelihood that the patient will respond to Compound 1 treatment, can be diagnosed by the practice of the described methods in which the expression levels of genes within the gene signatures are measured. In various embodiments, the expression levels of at least three, at least four, or all, of the genes of FIG. 1 are measured; or the expression of at least one, at least two, at least three, or at least four, or all, of the gene signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC are measured; at least one, at least two, at least three, at least four, or all, of the genes consisting of MDM2, CDKN1A, ZMAT3, DDB2, FDXR, RPS27L, BAX, RRM2B, SESN1, CCNG1, XPC, TNFRSF10B and AEN are measured; at least one, at least two, at least three, at least four, or all, of the genes consisting of BAX, RPS27L, EDA2R, XPC, DDB2, FDXR, MDM2, CDKN1A, TRIAP1, BBC3, CCNG1, TNFRSF10B, and CDKN2A are measured; or the expression of at least one, at least two, at least three, or all, of the gene signature genes consisting of RPS27L, FDXR, CDKN1A and AEN are measured. By example, expression of gene signature genes in a patient's cancer or tumor sample undergoing testing and indicative of Compound 1 sensitivity serves to diagnose the patient as one who will be sensitive to Compound 1 treatment.

As used herein, “differentially expressed” refers to a difference or alteration in expression, such as an increase or a decrease, in the conversion of gene-encoded information, (such as a gene associated with Compound 1 sensitivity), into RNA (e.g., mRNA), and/or in the conversion of mRNA into protein. In some cases, the difference or alteration is relative to a control or a reference value, or to a range of control or reference values, for example, the average expression of a group or a population of subjects, such as a group of subjects having a good response or a poor response to Compound 1 treatment (e.g., Compound 1 sensitive versus Compound 1 insensitive populations). In some cases, the difference or alteration can be relative to non-tumor tissue from the same subject or a healthy subject. The detection of differential expression can involve measuring a change in gene or protein expression, such as a change in expression of at least three, or at least four of the gene signature genes of FIG. 1 associated with Compound 1 sensitivity.

Detecting the expression of a gene product, as well as detecting the differential expression of a gene product, refer to measuring, or determining qualitatively or quantitatively, the level of expression of nucleic acid or protein in a sample by one or more suitable means as known in the art, e.g., by microarray analysis, PCR (RT-PCR), immunohistochemistry, immunofluorescence, mass spectrometry, Northern blot, Western blot, etc.

The term “prognosis” refers to the prediction of prospective survival and recovery from a disease or condition, as anticipated from the usual course of that disease or condition, or as indicated by special features presented by a subject. A prognosis can also predict the course of a disease associated with a particular treatment, for example, by determining that a patient will or will be likely to survive for a given period of time, depending on, for example, a patient's response or sensitivity to a given therapy or treatment regimen involving one or more drugs or compounds. Thus, the practice of the methods of the invention in which the sensitivity of a patient's AML to Compound 1 is determined by measuring expression levels of genes of the described Compound 1 sensitive gene signatures is associated with a prognosis that the patient will respond, or is likely to respond, to Compound 1 treatment. In various embodiments, the expression levels of at least one, at least two, at least three, at least four, or all, of the genes of FIG. 1 are measured; or the expression of at least one, at least two, at least three, at least four, or all, of the gene signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC are measured; at least one, at least two, at least three, at least four, or all, of the genes consisting of MDM2, CDKN1A, ZMAT3, DDB2, FDXR, RPS27L, BAX, RRM2B, SESN1, CCNG1, XPC, TNFRSF10B and AEN are measured; at least one, at least two, at least three, at least four, or all, of the genes consisting of BAX, RPS27L, EDA2R, XPC, DDB2, FDXR, MDM2, CDKN1A, TRIAP1, BBC3, CCNG1, TNFRSF10B, and CDKN2A are measured; or the expression of at least one, at least two, at least three, or all, of the gene signature genes consisting of RPS27L, FDXR, CDKN1A and AEN are measured.

As referred to herein, “sensitivity to treatment” relates to AML that is responsive to an initial, and in some cases, a subsequent or ongoing, therapy or treatment. Sensitivity may refer to the responsiveness of a disease, symptom, or progression of AML, such as the growth of AML or AML cells, to Compound 1. For example, an increased (relative) sensitivity refers to a state in which AML is more responsive to a given therapy or therapeutic agent or treatment as compared to AML that is not sensitive to the treatment.

The term “pharmaceutically acceptable salt” refers to salts of the active compounds which are relatively nontoxic acid or base addition salts. Non-limited examples of the acid addition salts include hydrochloric, hydrobromic, nitric, carbonic, phosphoric, acetic, propionic, isobutyric, maleic, malonic, benzoic, succinic, fumaric, lactic, benzenesulfonic, p-toluenesulfonic, citric, tartaric, oxalic, and methanesulfonic acids. The term “pharmaceutically acceptable salt” includes pharmaceutically acceptable solvate or salt thereof. The solvate is a stoichiometric complex of a molecule and one or more solvent molecules. Non-limited examples of pharmaceutically acceptable solvates include water, methanol, ethanol, dimethylsulfoxide, and acetate as solvent. A solvate which contains water as solvent is hydrate. In a preferable embodiment of the invention, the pharmaceutically acceptable salt of the compound can be hydrate and more preferably monohydrate.

The term “about” used herein refers to the specific value subsequent to the term and a range of values ±10% of the specific value. For example, the phrase “about 100” refers to 100, which is the specific value in this case, and a range of 90 to 110.

The compound of the formula (I) and pharmaceutically acceptable salts thereof, including the p-toluenesulfonate salt thereof, are disclosed as one of MDM2 inhibitors (see, Example 70 of WO 2012/121361 and Example 70 of US Patent Application Publication No. 2012/0264738A).

In a preferable embodiment of the invention, the salt of the compound of formula (I) can be the compound of formula (II):

(3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrro lidine-3′,3″-indole]-5′-carboxamide mono(4-methylbenzenesulfonate) monohydrate (also referred to as “mono p-toluenesulfonate monohydrate of the compound of formula (I)” or as “Compound 2”).

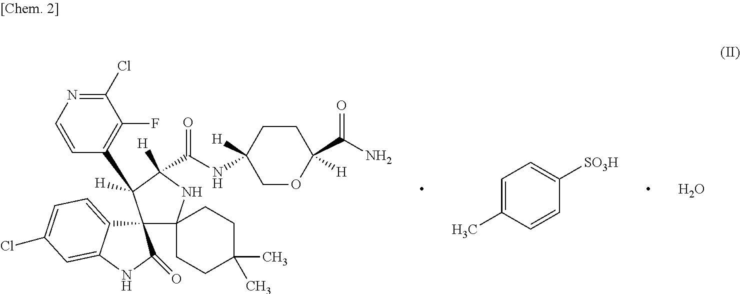 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21 ; Cc1ccc(S(=O)(=O)O)cc1 ; O]

Compound 1 or 2 can be administered once daily to a patient suffering from AML such as newly diagnosed, relapsed or refractory AML in order to treat AML in the patient.

Compound 1 or 2 can act as an MDM2 inhibitor. MDM2 is a negative regulator of the p53 tumor suppressor protein. The 90 kDa MDM2 protein contains a p53 binding domain at its N-terminus and a RING (really interesting gene) domain at its C-terminus, which functions as an E3 ligase that ubiquitinates p53. The activation of wild-type p53 by cell stimuli and stresses results in the binding of MDM2 to p53 at the N-terminus to inhibit the transcriptional activation of p53 and promote the degradation of p53 via the ubiquitin-proteasome pathway. Thus, MDM2 can interfere with p53-mediated apoptosis and arrest of cancer cell proliferation, attributing a significant oncogenic activity to MDM2 in cancer cells. In some cases, MDM2 can cause carcinogenesis independent of the p53 pathway, for example, in cells which possess an alternative splice form of MDM2. (H. A. Steinman et al., 2004, J. Biol. Chem., 279(6):4877-4886). In addition, about 50% of human cancers are observed to have a mutation in or deletion of the TP53 gene. MDM2 is overexpressed in a number of human cancers, including, for example, melanoma, non-small cell lung cancer (NSCLC), breast cancer, esophageal cancer, leukemia, non-Hodgkin's lymphoma and sarcoma.

Therefore, it is preferable that AML to be treated in the invention has amplified MDM2 genes on the genome of the subject suffering from AML or have activated MDM2 in AML. In a specific embodiment of the invention, AML to be treated in the invention can be AML with amplified MDM2 genes on the genome of the AML.

It is also preferable that AML to be treated in the invention have wild-type TP53 gene on the genome of the AML. In a specific embodiment of the invention, AML to be treated in the invention can be AML which has one or more wild-type TP53 genes on the genome of the AML.

In a more specific embodiment of the invention, AML to be treated in the invention can be AML which has wild-type TP53 gene and amplified MDM2 genes on the genome of the AML.

In these specific embodiments, the AML which has wild-type TP53 gene and/or amplified MDM2 genes on the genome of the AML can effectively be treated by the administration of Compound 1 or 2.

The inventors have discovered that the gene signatures predictive of MDM2i sensitivity as disclosed in WO2015/108175 are also useful in predicting the sensitivity of AML to Compound 1 or 2. The 177 genes shown in FIG. 1 were identified from the data obtained from a multi-cancer cell line panel in WO2015/108175. The expression of each of the 177 genes positively correlates to the sensitivity of cancers to MDM2i treatment. WO2015/108175 demonstrated that the gene signature of the 177 genes is predictive of a cancer or tumor sample's sensitivity to an MDM2i. Also, 40 genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC, which are included in the 177 genes, were also shown as signature genes predictive of MDM2i sensitivity in WO2015/108175.

Even the four genes: RPS27L, FDXR, CDKN1A and AEN among the 177 genes were established as gene signatures which are useful in predicting MDM2i sensitivity of cancers in WO2015/108175, and can be used as gene signature in the invention. At least four or all of the genes: MDM2, CDKN1A, ZMAT3, DDB2, FDXR, RPS27L, BAX, RRM2B, SESN1, CCNG1, XPC, TNFRSF10B and AEN, preferably including the above four signature genes, can also be used as gene signature in the invention. In another embodiment of the invention, at least four or all of the genes: BAX, RPS27L, EDA2R, XPC, DDB2, FDXR, MDM2, CDKN1A, TRIAP1, BBC3, CCNG1, TNFRSF10B, and CDKN2A, preferably including the above four signature genes, can also be used as gene signature in the invention.

The present inventors have discovered that the sensitivity of AML to MDM2i such as Compound 1 or 2 can also be predicted by using the signature genes. The present inventors have also discovered that the sensitivity of AML to MDM2i such as Compound 1 or 2 can also be predicted by using both of the signature genes and the TP53 genotype.

Therefore, the present invention provides a method of predicting sensitivity to Compound 1 or 2 treatment in a patient suffering from AML, comprising measuring the expression levels of at least one, at least two, at least three or all of the four genes: RPS27L, FDXR, CDKN1A and AEN. The invention provides a method of predicting sensitivity to Compound 1 or 2 treatment in a patient suffering from AML, comprising measuring the expression levels of at least one, at least two, at least three, at least four or all of the genes below: MDM2, CDKN1A, ZMAT3, DDB2, FDXR, RPS27L, BAX, RRM2B, SESN1, CCNG1, XPC, TNFRSF10B and AEN, the genes to be measured preferably comprising the genes: RPS27L, FDXR, CDKN1A and AEN. The invention provides a method of predicting sensitivity to Compound 1 or 2 treatment in a patient suffering from AML, comprising measuring the expression levels of at least one, two, three, four or all of the genes below: BAX, RPS27L, EDA2R, XPC, DDB2, FDXR, MDM2, CDKN1A, TRIAP1, BBC3, CCNG1, TNFRSF10B, and CDKN2A, the genes to be measured preferably comprising the genes: RPS27L, FDXR, CDKN1A and AEN. The invention provides a method of predicting sensitivity to Compound 1 or 2 treatment in a patient suffering from AML, comprising measuring the expression levels of at least one, at least two, at least three, at least four or all of the forty signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC, the genes to be measured preferably comprising the genes: RPS27L, FDXR, CDKN1A and AEN. The invention provides a method of predicting sensitivity to Compound 1 or 2 treatment in a patient suffering from AML, comprising measuring the expression levels of at least one, at least two, at least three, at least four or all of the 175 signature genes (i.e., the genes presented in FIG. 1, except for EDA2R and SPATA18), the genes to be measured preferably comprising the genes: RPS27L, FDXR, CDKN1A and AEN. The invention provides a method of predicting sensitivity to Compound 1 or 2 treatment in a patient suffering from AML, comprising measuring the expression levels of at least one, at least two, at least three, at least four or all of the 177 signature genes shown in FIG. 1, the genes to be measured preferably comprising the genes: RPS27L, FDXR, CDKN1A and AEN. In a preferred embodiment, the invention provides a method of predicting sensitivity to Compound 1 or 2 treatment in a patient suffering from AML, comprising determining the genotype of TP53 gene and measuring the expression levels of at least one, at least two, at least three, at least four or all of the signature genes described above. In a specific embodiment, the invention provides a method of predicting sensitivity to Compound 1 or 2 treatment in a patient suffering from AML, comprising determining the genotype of TP53 gene, and measuring the expression levels of at least one, at least two, at least three, at least four or all of the signature genes described above, when the patient has mutant TP53 gene or the patient has wild-type TP53 gene and has a low signature score compared to the predetermined cutoff value, then the AML is predicted as resistant and when the patient has wild-type TP53 gene and has a high signature score compared to the predetermined cutoff value, then the AML is predicted as sensitive.

In some cases, expression levels of the signature genes (such as expression of at least three, at least four, at least five, at least six, at least ten, or all, of the genes listed in the gene signatures of FIG. 1; at least three, or all, of the genes in the gene set consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC; at least one, at least two, at least three, or all, of the genes in the gene set MDM2, CDKN1A, ZMAT3, DDB2, FDXR, RPS27L, BAX, RRM2B, SESN1, CCNG1, XPC, TNFRSF10B and AEN; at least one, at least two, at least three, or all, of the genes in the gene set BAX, RPS27L, EDA2R, XPC, DDB2, FDXR, MDM2, CDKN1A, TRIAP1, BBC3, CCNG1, TNFRSF10B, and CDKN2A; or at least one, at least two, at least three, or all, of the genes in the gene set RPS27L, FDXR, CDKN1A and AEN) are normalized relative to the expression levels of one or more housekeeping genes, e.g., in the same or different cancer or neoplasm sample. An aggregate value is obtained in some cases by calculating the level of expression of each of the genes (e.g., each of the genes in a gene signature) and using a positive or negative weighting for each gene depending on whether the gene is positively or negatively regulated by a condition (e.g., sensitivity to Compound 1 or 2 treatment or a survival risk score). In some cases, the normalized expression of the gene or the gene signature, or an aggregate value, is determined to be increased or decreased relative to the median normalized expression of the gene or gene signature, or to an aggregate value, for AML. In some cases, the median normalized expression or aggregate value is obtained from publicly available microarray datasets, such as leukemia, lymphoma, melanoma, or myeloma cancer microarray datasets. In an example, a median normalized expression or aggregate value for expression genes of the gene signature is determined using microarray datasets.

In an embodiment, the use of a sensitivity score can be advantageous, as the score can be used as the basis for defining whether AML is sensitive to Compound 1 or 2 and can thus be predictive that an individual having an AML sensitive to the compound will respond favorably to the treatment with the compound. For example, upon the determination of a sensitivity score indicative of AML sample's sensitivity to Compound 1 or 2, a medical practitioner may elect to treat a patient having the AML with Compound 1 or 2. Alternatively, upon the determination of a sensitivity score indicative of AML sample's insensitivity to Compound 1 or 2, a medical practitioner may elect not to treat a patient having the AML with Compound 1 or 2, as the patient would be predicted not to receive a clinical or medical benefit from the treatment with Compound 1 or 2. If a sample of a patient's AML is assessed for the sensitivity to Compound 1 or 2 during a course of a treatment or therapy with Compound 1 or 2, a sensitivity score indicative of the sensitivity to Compound 1 or 2 may assist the medical practitioner in deciding to continue or alter the patient's AML treatment or therapy and/or to treat with Compound 1 or 2.

In some cases, a sensitivity score is calculated from the normalized expression level measurements. The score can be utilized to provide cutoff points or values to identify various parameters, such as AML as being sensitive, or less likely to be sensitive, to Compound 1 or 2 and/or low, medium, or high sensitivity of a patient with AML to the treatment or therapy with Compound 1 or 2. In some cases, the cutoff points are often determined using training and validation datasets. By way of example, a supervised approach can be utilized to establish the cutoff that distinguishes those who will be sensitive (responders) from those who will not respond to Compound 1 or 2 treatment, for example, by comparing gene signature expression in responders and non-responders. In another example, an unsupervised approach can be utilized to determine empirically a cutoff level (for example, top 50% versus bottom 50%, top quartile versus bottom quartile or top tercile versus bottom tercile) that is predictive of an outcome, i.e., sensitivity to Compound 1 or 2 treatment. The cutoff determined in the training set can be tested in one or more independent validation datasets. The cutoff values can be determined or adjusted, considering a desirable false-positive rate and false-negative rate. In an embodiment, the cutoff values can be equal to or more than the median signature score of a group of patients suffering from AML, preferably the signature score is a sum of the Z-score of the expression level of each of the above signature genes. In an embodiment, the cutoff values can be equal to or more than the first quartile value (Q_{1/4}) of the signature scores of a group of patients suffering from AML, preferably the signature score is the unweighted or weighted average of the Z-score of the expression level of each of the above signature genes. In an embodiment, the cutoff values can be equal to or more than the third quartile value (Q_{3/4}) of the signature scores of a group of patients suffering from AML, preferably the signature score is the unweighted or weighted average of the Z-score of the expression level of each of the above signature genes. In an embodiment, the cutoff values can be in interquartile range of the signature scores of a group of patients suffering from AML, preferably the signature score is the unweighted or weighted average of the Z-score of the expression level of each of the above signature genes.

In another aspect of the invention, the invention provides a pharmaceutical composition for use in treating AML in a patient in need thereof, comprising Compound 1 or 2, wherein the patient has been predicted as sensitive by a method of predicting sensitivity as described above. In a specific embodiment, the invention provides a pharmaceutical composition for use in treating AML in a patient in need thereof, comprising Compound 1 or 2, wherein the patient has been predicted as sensitive by a method of predicting sensitivity to the compound treatment in the patient, the method comprising measuring the expression levels of at least one, at least two, at least three or all of the four genes: RPS27L, FDXR, CDKN1A and AEN in a sample obtained from the patient. In another specific embodiment, the invention provides a pharmaceutical composition for use in treating AML in a patient in need thereof, comprising Compound 1 or 2, wherein the patient has been predicted as sensitive by a method of predicting sensitivity to the compound treatment in the patient, the method comprising measuring the expression levels of at least one, at least two, at least three, four or all of the genes below: MDM2, CDKN1A, ZMAT3, DDB2, FDXR, RPS27L, BAX, RRM2B, SESN1, CCNG1, XPC, TNFRSF10B and AEN in a sample obtained from the patient, the genes to be measured preferably comprising the genes: RPS27L, FDXR, CDKN1A and AEN. In another specific embodiment, the invention provides a pharmaceutical composition for use in treating AML in a patient in need thereof, comprising Compound 1 or 2, wherein the patient has been predicted as sensitive by a method of predicting sensitivity to the compound of the compound treatment in the patient, the method comprising measuring the expression levels of at least one, at least two, at least three, four or all of the genes below: BAX, RPS27L, EDA2R, XPC, DDB2, FDXR, MDM2, CDKN1A, TRIAP1, BBC3, CCNG1, TNFRSF10B, and CDKN2A in a sample obtained from the patient, the genes to be measured preferably comprising the genes: RPS27L, FDXR, CDKN1A and AEN. In another specific embodiment, the invention provides a pharmaceutical composition for use in treating AML in a patient in need thereof, comprising Compound 1 or 2, wherein the patient has been predicted as sensitive by a method of predicting sensitivity to the compound treatment in the patient, the method comprising measuring the expression levels of at least one, at least two, at least three, four or all of the 40 signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in a sample obtained from the patient, the genes to be measured preferably comprising the genes: RPS27L, FDXR, CDKN1A and AEN. In another specific embodiment, the invention provides a pharmaceutical composition for use in treating AML in a patient in need thereof, comprising Compound 1 or 2, wherein the subject has been predicted as sensitive by a method of predicting sensitivity to the compound treatment in the patient, the method comprising measuring the expression levels of at least one, at least two, at least three, four or all of the 175 signature genes (i.e., the genes presented in FIG. 1, except for EDA2R and SPATA18) in a sample obtained from the patient, the genes to be measured preferably comprising the genes: RPS27L, FDXR, CDKN1A and AEN. In another specific embodiment, the invention provides a pharmaceutical composition for use in treating AML in a patient in need thereof, comprising Compound 1 or 2, wherein the patient has been predicted as sensitive by a method of predicting sensitivity to the compound treatment in the patient, the method comprising measuring the expression levels of at least one, at least two, at least three, four or all of the 177 signature genes shown in FIG. 1 in a sample obtained from the patient, the genes to be measured preferably comprising the genes: RPS27L, FDXR, CDKN1A and AEN.

In another aspect of the invention, the invention provides a method for treating AML in a patient in need thereof, comprising administering Compound 1 or 2 to the patient. In an embodiment of the invention, the invention provides a method for treating AML in a patient in need thereof, comprising administering Compound 1 or 2 to the patient, wherein the patient has been predicted as sensitive by any one of the methods of predicting sensitivity to Compound 1 or 2 in the subject as described above.

A pharmaceutical composition of the present invention can contain Compound 1 or 2 and a pharmaceutically acceptable carrier, and can be administered as various injections such as intravenous injection, intramuscular injection, and subcutaneous injection or by various methods such as oral administration or percutaneous administration. “Pharmaceutically acceptable carrier” means a pharmacologically acceptable material that is involved in transport of Compound 1 or or a composition containing Compound 1 or 2 (for example, an excipient, a diluent, an additive, a solvent, etc.) from a given organ to another organ.

A formulation can be prepared by selecting a suitable formulation form (for example, oral formulation or injection) depending on the administration method and using various conventionally used methods for preparing a formulation. Examples of oral formulations include tablets, powders, granules, capsules, pills, lozenges, solutions, syrups, elixirs, emulsions, oily or aqueous suspensions, and so forth. In oral administration, the free compound or a salt form may be used. An aqueous formulation can be prepared by forming an acid adduct with a pharmacologically acceptable acid or by forming an alkali metal salt such as sodium. As an injection, a stabilizer, a preservative, a dissolving aid, and the like can be used in the formulation. After filling a solution that may contain these aids and the like in a vessel, a formulation for use may be prepared as a solid formulation by lyophilization or the like. Furthermore, one dose may be filled in one vessel, or two or more doses may be filled in a vessel.

Examples of solid formulations include tablets, powders, granules, capsules, pills, and lozenges. These solid formulations may contain pharmaceutically acceptable additives together with Compound 1 or 2. Examples of additives include fillers, extenders, binders, disintegrating agents, dissolution promoting agents, skin wetting agents, and lubricants, and these can be selected and mixed as required to prepare a formulation.

Examples of liquid formulations include solutions, syrups, elixirs, emulsions, and suspensions. These liquid formulations may contain pharmaceutically acceptable additives together with Compound 1 or 2. Examples of additives include suspending agents and emulsifiers, and these are selected and mixed as required to prepare a formulation.

Compound 1 or 2 can be used in AML treatment of mammals, in particular, humans. The dose and the administration interval can be suitably selected depending on the site of the disease, the patient's height, body weight, sex, or medical history, according to a physician's judgment. When Compound 1 or 2 is administered to a human, the dose range is approx. 0.01 to 500 mg/kg body weight per day, preferably, approx. 0.1 to 100 mg/kg body weight. Preferably, Compound 1 or 2 is administered to a human once a day, or the dose is divided two to four times, and administration is repeated at an appropriate interval. Furthermore, the daily dose may exceed the above-mentioned dose at a physician's discretion, if necessary.

Compound 1 or 2 may be used in combination with additional anti-tumor agent(s). Examples thereof include anti-tumor antibiotics, anti-tumor plant constituents, BRMs (biological response modifiers), hormones, vitamins, anti-tumor antibodies, molecular target drugs, and other anti-tumor agents such as an MDM2i.

More specifically, examples of alkylating agents include the following: alkylating agents such as nitrogen mustard, nitrogen mustard N-oxide, bendamustine and chlorambucil; amidine alkylating agents such as carboquone and thiotepa; epoxide alkylating agents such as dibromomannitol and dibromodulcitol; nitrosourea alkylating agents such as carmustine, lomustine, semustine, nimustine hydrochloride, streptozocin, chlorozotocin, and ranimustine; and busulfan, improsulfan tosylate, and dacarbazine.

Examples of various metabolic antagonists include the following: purine metabolic antagonists such as 6-mercaptopurine, 6-thioguanine, and thioinosine; pyrimidine metabolic antagonists such as fluorouracil, tegafur, tegafur-uracil, carmofur, doxifluridine, broxuridine, cytarabine, and enocitabine; and folic acid metabolic antagonists such as methotrexate and trimetrexate.

Examples of anti-tumor antibiotics include mitomycin C, bleomycin, peplomycin, daunorubicin, aclarubicin, doxorubicin, idarubicin, pirarubicin, THP-adriamycin, 4′-epidoxorubicin, and epirubicin; and chromomycin A3 and actinomycin D.

Examples of anti-tumor plant constituents and their derivatives include the following: vinca alkaloids such as vindesine, vincristine, and vinblastine; taxanes such as paclitaxel, docetaxel, and cabazitaxel; and epipodophyllotoxins such as etoposide and teniposide.

Examples of BRMs include tumor necrosis factors and indomethacin.

Examples of hormones include hydrocortisone, dexamethasone, methylprednisolone, prednisolone, prasterone, betamethasone, triamcinolone, oxymetholone, nandrolone, metenolone, fosfestrol, ethinylestradiol, chlormadinone, medroxyprogesterone, and mepitiostane.

Examples of vitamins include vitamin C and vitamin A.

Examples of anti-tumor antibodies and molecular target drugs include trastuzumab, rituximab, cetuximab, nimotuzumab, denosumab, bevacizumab, infliximab, ipilimumab, nivolumab, pembrolizumab, avelumab, pidilizumab, atezolizumab, ramucirumab imatinib mesilate, dasatinib, gefitinib, erlotinib, sunitinib, lapatinib, vemurafenib, dabrafenib, trametinib, pazopanib, palbociclib, panobinostat, sorafenib, ibrutinib, bortezomib, carfilzomib, ixazomib, and quizartinib.

Examples of other anti-tumor agents include cisplatin, carboplatin, oxaliplatin, tamoxifen, letrozole, anastrozole, exemestane, toremifene citrate, fulvestrant, bicalutamide, flutamide, mitotane, leuprorelin, goserelin acetate, camptothecin, ifosfamide, cyclophosphamide, melphalan, L-asparaginase, aceglatone, sizofuran, picibanil, procarbazine, pipobroman, neocarzinostatin, hydroxyurea, ubenimex, azacytidine, decitabine, thalidomide, lenalidomide, pomalidomide, eribulin, tretinoin, and krestin.

Hereinafter, the following examples are provided only for illustrative purposes and it is understood that the invention is not limited the examples.

### EXAMPLES

### Example 1

Leukemia samples isolated from peripheral blood or bone marrow of patients with newly diagnosed or relapsed/refractory AML were treated using the test compound as described below.

Test Compound

(3′R,4′S,5′R)—N-[(3R,6S)-6-carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide mono(4-methylbenzenesulfonate) monohydrate (Compound 2) was prepared as described in WO2014/038606.

Leukemia Samples

Heparinized peripheral blood and bone marrow samples containing more than 1.6×10⁷ mononuclear cells were obtained from AML patients with newly diagnosed or relapsed/refractory AML, after informed consent according to the University of Texas MD Anderson Cancer Center (MDA, Houston, Tex., USA) guidelines in accordance with the Declaration of Helsinki. In each sample, the blast counts (blast %) was determined from routine morphological differential counts by clinical laboratory technicians, confirmed by hematopathologists at MDA (usually 500 cells were counted). The bone marrows or the peripheral blood cells with more than 50% blasts were selected and used as AML samples in the Example. High percentages (>30%) of spontaneous apoptosis was observed in three samples among the forty-four samples, which were excluded for this purpose. Characteristics of the AML samples are shown in Table 1.

TABLE 1
Summary of the patients to be analyzed

| Sample No. | Age | Sex | Source | Blast % |
|---|---|---|---|---|
| 1 | 77 | M | BM | 86 |
| 2 | 76 | M | PB | 62 |
| 3 | 50 | M | PB | 50 |
| 4 | 40 | M | PB | 93 |
| 5 | 84 | F | PB | 63 |
| 6 | 55 | M | PB | 95 |
| 7 | 35 | F | PB | 63 |
| 8*¹ | 51 | F | PB | 54 |
| 9*¹ | 51 | F | BM | 67 |
| 10 | 76 | M | PB | 93 |
| 11 | 74 | M | PB | 51 |
| 12 | 54 | F | BM | 82 |
| 13 | 77 | M | PB | 92 |
| 14 | 42 | F | PB | 99 |
| 15 | 83 | M | PB | 80 |
| 16 | 66 | M | PB | 75 |
| 17 | 52 | F | PB | 68 |
| 18 | 69 | F | PB | 88 |
| 19 | 47 | F | PB | 95 |
| 20 | 30 | F | PB | 89 |
| 21 | 78 | M | PB | 64 |
| 22 | 32 | M | BM | 83 |
| 23 | 57 | M | PB | 59 |
| 24 | 76 | M | PB | 80 |
| 25 | 85 | F | BM | 51 |
| 26 | 87 | F | BM | 68 |
| 27 | 72 | M | BM | 67 |
| 28 | 52 | M | PB | 90 |
| 29 | 76 | F | PB | 75 |
| 30 | 64 | M | PB | 99 |
| 31 | 90 | M | PB | 70 |
| 32 | 29 | M | PB | 72 |
| 33*² | 72 | M | BM | 89 |
| 34*² | 72 | M | PB | 93 |
| 35 | 78 | M | PB | 66 |
| 36 | 73 | M | PB | 78 |
| 37 | 69 | F | PB | 83 |
| 38 | 69 | M | BM | 51 |
| 39 | 27 | F | PB | 51 |
| 40 | 48 | M | PB | 70 |
| 41 | 77 | M | PB | 91 |
| 42 | 77 | M | BM | 97 |
| 43*³ | 69 | M | PB | 84 |
| 44*³ | 69 | M | BM | 74 |

M: Male;
F: Female;
PB: peripheral blood;
BM: bone marrow;
*¹, *² and *³the samples indicated with the same annotation are derived from the same patient.

TP53 Genotyping

Genomic DNA (gDNA) was extracted from bone marrow aspirates or peripheral blood of each case using an Autopure extractor (Qiagen, Valencia, Calif.) and was quantified using a Qubit DNA BR assay kit (Life Technologies, Carlsbad, Calif.). The genomic library was prepared using 250 ng of DNA template and a commercially available 48-gene TruSeq Amplicon Cancer Panel (Illumina Inc., San Diego, Calif.) to which custom-designed probe pairs for five genes were added. The 53-gene panel, including TP53, was published previously. (Ok et al, Leukemia Research (2015) 39, 348-354) The generated library was purified using AMPure magnetic beads (Agencourt, Brea, Calif.) and then subjected to next-generation sequencing using a MiSeq sequencer (Illumina Inc., SanDiego, Calif.). Sanger sequencing was performed to confirm mutations in TP53.

Detection of Apoptosis

Mononuclear cells were purified by density-gradient centrifugation. The cells were cultured in RPMI 1640 medium containing 10% fetal bovine serum and were treated ex vivo for 48 hours with the test compound (0, 25, 50, 100, 250, 500, or 1000 nM), and live cell numbers were determined by apoptosis analysis using annexin V and propidium iodide (PI, purchased from Sigma-Aldrich) binding assays. Annexin V- and PI-negative cells were counted as live cells. Apoptosis was quantified as the proportion of annexin V-positive cells, and specific apoptosis was calculated by the following formula: % specific apoptosis=(test−control)×100/(100−control).

AUC % Live Cells

To define drug sensitivity/resistance, area under the curve (AUC) values were calculated so that sensitive samples would have relatively low AUC values and resistant samples would have relatively large AUC values. In particular, the AUCs were calculated using the R software ver. 3.2.0 provided by R Development Core Team by approximating the integral of function by calculating the area under a smoothed spline fitted to the given x and y(x) values {x: a concentration of Compound 2, y(x): the measured % live cells}.

Determination of Sensitivity

Among 33 cases with wild-type TP53, 11 samples each were determined as sensitive (low AUC group) or resistant (high AUC group) to the test compound based on AUC values. In the genotype mixed samples, 14 samples each were selected as sensitive or resistant to the test compound based on AUC values.

Gene Signature

Baseline whole-genome RNA expression profile (Affymetrix Human Genome U133 Plus 2.0 Array) of the 41 AML samples was determined. The RNA was amplified using the 3′ IVT Express kit from Affymetrix. The RNA (100 ng) was reverse transcribed to synthesize first strand cDNA. This cDNA was then converted into a double stranded DNA template for transcription to generate aRNA (cRNA) and incorporate a biotin conjugated nucleotide, fragmented for hybridization on the Human Gene U133_2.0 array. The arrays were processed using the Affymetrix GeneChip Hybridization, Washing and Staining kit, using the Affymetrix Fluidics station 450 controlled by Affymetrix GeneChip Command Console (AGCC) Software. The arrays were scanned using the Affymetrix GeneChip Scanner 3000, 7G controlled by AGCC Software. The arrays were analyzed with the MAS5 algorithm using the Affymetrix Expression Console default analysis settings.

The 175-gene signature or 40-gene signature as established in a wide range of cancer cells in WO2015/108175 was applied to the resistant or sensitive samples. In the 175-gene signature, the mRNA expression profile of the 175 genes (i.e., the genes presented in FIG. 1, except for EDA2R and SPATA18) was determined. In the 40-gene signature, the mRNA expression profile of the 40 genes (i.e., the gene consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC was determined.

To normalize the contribution of each gene in the signature, signature genes were subject to Z-score normalization (the mean expression value of each gene is subtracted from the value within each sample, and the difference is divided by the standard deviation). Sensitivity scores (Signature scores) were generated by determining the unweighted average of the Z-score normalized expression values of each signature gene.

### Results

Apoptosis of AML was induced with the test compound in the samples. The results of the % live cell number (compared to untreated well) were shown in Table 2.

TABLE 2
% live cell number (compared to untreated well)

| Sam- | | | | | | | |
|---|---|---|---|---|---|---|---|
| ple | | | | | | | |
| No. | 0 nM | 25 nM | 50 nM | 100 nM | 250 nM | 500 nM | 1000 nM |
| 1 | 100 | 92.25 | 110.61 | 71.51 | 100.38 | 89.73 | 105.81 |
| 2 | 100 | 104.13 | 97.67 | 78.084 | 48.20 | 31.85 | 21.45 |
| 3 | ND | ND | ND | ND | ND | ND | ND |
| 4 | 100 | 128.88 | 67.95 | 93.04 | 61.53 | 48.72 | 52.86 |
| 5 | 100 | 81.43 | 106.29 | 164.24 | 117.18 | 94.30 | 119.74 |
| 6 | 100 | 66.91 | 65.33 | 92.36 | 74.82 | 47.20 | 20.88 |
| 7 | 100 | 103.00 | 77.57 | 74.74 | 62.00 | 49.81 | 34.54 |
| 8 | 100 | 90.92 | 71.19 | 58.73 | 44.46 | 39.35 | 46.57 |
| 9 | 100 | 98.88 | 47.14 | 37.00 | 32.55 | 31.37 | 20.14 |
| 10 | 100 | 73.78 | 75.72 | 46.18 | 10.64 | 3.14 | 2.71 |
| 11 | 100 | 146.77 | 72.18 | 39.13 | 56.69 | 42.18 | 36.45 |
| 12 | 100 | 158.06 | 92.42 | 104.02 | 86.50 | 65.75 | 47.79 |
| 13 | 100 | 127.54 | 127.78 | 175.35 | 174.61 | 128.80 | 189.06 |
| 14 | 100 | 34.04 | 35.94 | 12.93 | 3.84 | 5.19 | 5.06 |
| 15 | 100 | 83.71 | 78.42 | 72.55 | 57.83 | 37.16 | 41.36 |
| 16 | 100 | 55.94 | 83.72 | 79.15 | 83.26 | 94.19 | 94.20 |
| 17 | 100 | 58.22 | 79.80 | 72.15 | 42.95 | 43.00 | 23.02 |
| 18 | ND | ND | ND | ND | ND | ND | ND |
| 19 | 100 | 270.02 | 88.11 | 161.86 | 161.89 | 73.65 | 37.13 |
| 20 | 100 | 76.97 | 53.97 | 37.50 | 22.14 | 22.77 | 16.72 |
| 21 | 100 | 110.47 | 62.22 | 74.64 | 30.00 | 26.80 | 19.91 |
| 22 | 100 | 17.63 | 13.17 | 11.63 | 7.93 | 3.89 | 1.57 |
| 23 | 100 | 117.12 | 83.70 | 78.89 | 45.59 | 28.56 | 53.00 |
| 24 | 100 | 90.78 | 76.43 | 52.33 | 42.11 | 29.40 | 20.97 |
| 25 | 100 | 119.37 | 162.01 | 72.41 | 74.04 | 64.37 | 53.58 |
| 26 | 100 | 69.23 | 51.99 | 54.80 | 3.88 | 47.47 | 45.24 |
| 27 | 100 | 25.48 | 23.35 | 19.20 | 7.75 | 3.99 | 6.09 |
| 28 | 100 | 78.72 | 37.55 | 36.23 | 34.53 | 21.63 | 15.04 |
| 29 | 100 | 68.11 | 88.76 | 18.85 | 27.27 | 35.86 | 13.46 |
| 30 | 100 | 45.44 | 39.71 | 52.83 | 26.21 | 9.80 | 4.04 |
| 31 | 100 | 76.66 | 52.74 | 54.47 | 45.48 | 46.04 | 39.07 |
| 32 | 100 | 91.70 | 99.02 | 58.80 | 50.63 | 36.20 | 37.59 |
| 33 | 100 | 54.38 | 42.81 | 16.31 | 13.29 | 8.02 | 6.18 |
| 34 | 100 | 59.75 | 51.71 | 40.31 | 33.77 | 11.99 | 10.30 |
| 35 | 100 | 48.95 | 39.25 | 44.29 | 47.10 | 57.68 | 35.15 |
| 36 | 100 | 99.69 | 92.32 | 61.87 | 52.27 | 36.41 | 24.84 |
| 37 | 100 | 101.76 | 104.21 | 92.45 | 106.41 | 94.88 | 84.01 |
| 38 | 100 | 87.72 | 91.67 | 72.16 | 64.27 | 51.31 | 44.47 |
| 39 | 100 | 78.30 | 68.91 | 67.31 | 39.86 | 28.06 | 23.65 |
| 40 | 100 | 44.64 | 48.05 | 30.02 | 16.25 | 6.92 | 2.47 |
| 41 | 100 | 87.12 | 72.35 | 58.43 | 45.10 | 17.81 | 6.54 |
| 42 | ND | ND | ND | ND | ND | ND | ND |
| 43 | 100 | 23.99 | 7.36 | 6.54 | 17.11 | 3.90 | 2.74 |
| 44 | 100 | 34.32 | 13.40 | 6.14 | 7.14 | 4.13 | 2.10 |

ND: no data

Further, the results of AUC, TP53 status, and sensitivity to the test compound of the samples were summarized in Table 3. In Table 3, AUC of each sample was normalized with the AUC of Sample No. 13, which was the largest AUC among the samples.

TABLE 3
Summary of AUC, TP53 status and Sensitivity of each sample

| | Normalized | | |
|---|---|---|---|
| Sample No. | AUC | p53 status | Sensitivity |
| 1 | 0.569 | wild type | Resistant |
| 2 | 0.258 | wild type | — |
| 3 | NA | wild type | — |
| 4 | 0.376 | wild type | Resistant |
| 5 | 0.730 | V216M | Resistant |
| 6 | 0.277 | wild type | — |
| 7 | 0.315 | E285K | Resistant |
| 8 | 0.292 | wild type | — |
| 9 | 0.183 | wild type | — |
| 10 | 0.039 | wild type | Sensitive |
| 11 | 0.299 | wild type | Resistant |
| 12 | 0.447 | R110H | Resistant |
| 13 | 1 | D208V | Resistant |
| 14 | 0.003 | wild type | Sensitive |
| 15 | 0.298 | wild type | Resistant |
| 16 | 0.568 | R248W | Resistant |
| 17 | 0.258 | wild type | — |
| 18 | NA | Q192* | — |
| 19 | 0.595 | wild type | Resistant |
| 20 | 0.146 | wild type | Sensitive |
| 21 | 0.212 | wild type | — |
| 22 | 0.000 | wild type | Sensitive |
| 23 | 0.326 | wild type | Resistant |
| 24 | 0.213 | P8S | — |
| 25 | 0.378 | wild type | Resistant |
| 26 | 0.239 | wild type | — |
| 27 | 0.025 | N239_S240del | Sensitive |
| 28 | 0.143 | wild type | Sensitive |
| 29 | 0.134 | wild type | Sensitive |
| 30 | 0.087 | wild type | Sensitive |
| 31 | 0.265 | wild type | — |
| 32 | 0.245 | wild type | — |
| 33 | 0.060 | wild type | Sensitive |
| 34 | 0.118 | wild type | Sensitive |
| 35 | 0.274 | wild type | — |
| 36 | 0.261 | wild type | — |
| 37 | 0.565 | wild type | Resistant |
| 38 | 0.331 | R175H | Resistant |
| 39 | 0.215 | wild type | — |
| 40 | 0.034 | wild type | Sensitive |
| 41 | 0.164 | wild type | Sensitive |
| 42 | NA | wild type | — |
| 43 | 0.008 | wild type | Sensitive |
| 44 | 0.006 | wild type | Sensitive |

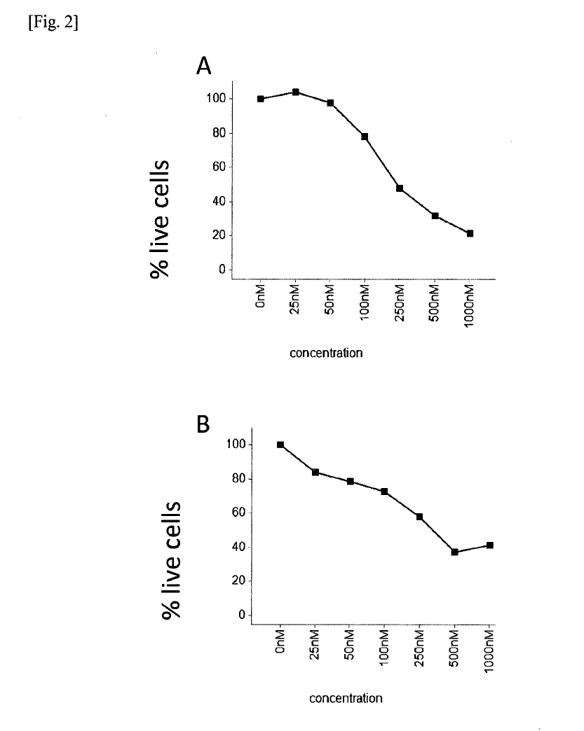

These results suggest that the test compound is useful in treating AML in a patient with newly diagnosed or relapsed/refractory AML. Further, in some of the samples, more than 80% of the leukemia cells induced apoptosis by the treatment of the test compound at a higher concentration (see FIG. 2 and Table 2), while, in other samples, only less than 20% of the leukemia cells induce apoptosis.

In order to determine whether or not the sensitivities of samples to the test compound are predictable, the samples were subjected to gene signature analysis using 175 genes or 40 genes as disclosed in a wide range of cancer cells in WO2015/108175.

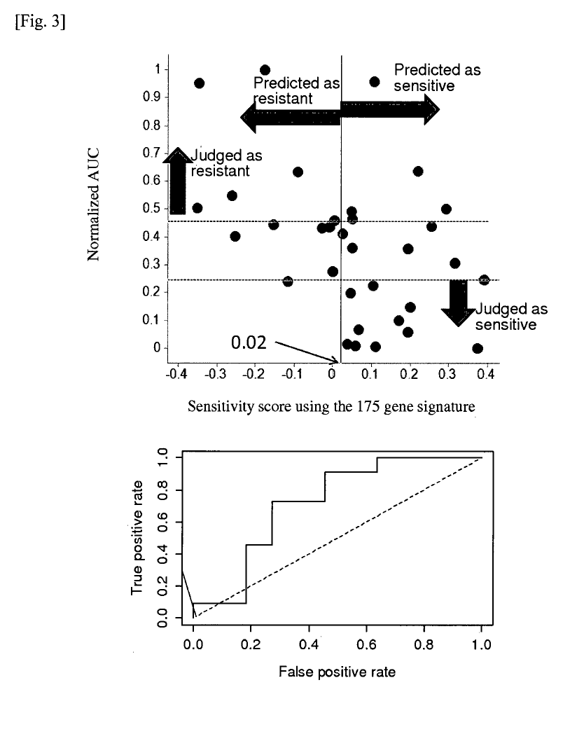

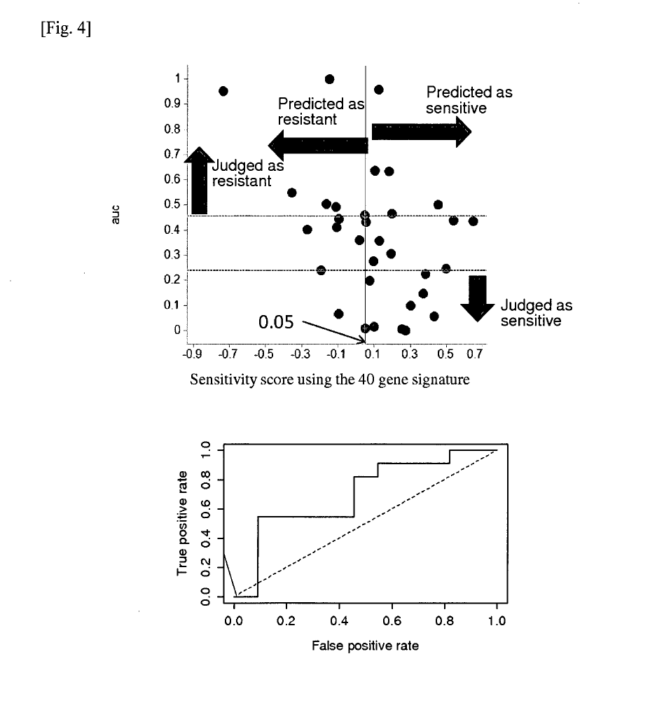

11 each p53 wild-type samples were selected as sensitive or resistant to the test compound based on AUC % live cells, and the 175-gene signature or 40-gene signature was applied. The prediction accuracy of the 175-gene signature was 72%, (see FIG. 3 and Table 4), when the cutoff value was 0.02. Also, the prediction accuracy of the 40-gene signature was 68%, (see FIG. 4 and Table 5), when the cutoff value was 0.05.

TABLE 4
Prediction performance of sensitivity (only TP53 wildtype samples)

| | Number of the samples | Number of the samples |
|---|---|---|
| | predicted as resistant | predicted as sensitive |
| Number of the samples | 6 | 5 |
| which are resistant | | |
| Number of the samples | 1 | 10 |
| which are sensitive | | |

The prediction accuracy: (6 + 10)/22 × 100% = 72%

TABLE 5
Prediction performance of sensitivity (only TP53 wildtype samples)

| | Number of the samples | Number of the samples |
|---|---|---|
| | predicted as resistant | predicted as sensitive |
| Number of the samples | 6 | 5 |
| which are resistant | | |
| Number of the samples | 2 | 9 |
| which are sensitive | | |

The prediction accuracy: (6 + 9)/22 × 100% = 68%

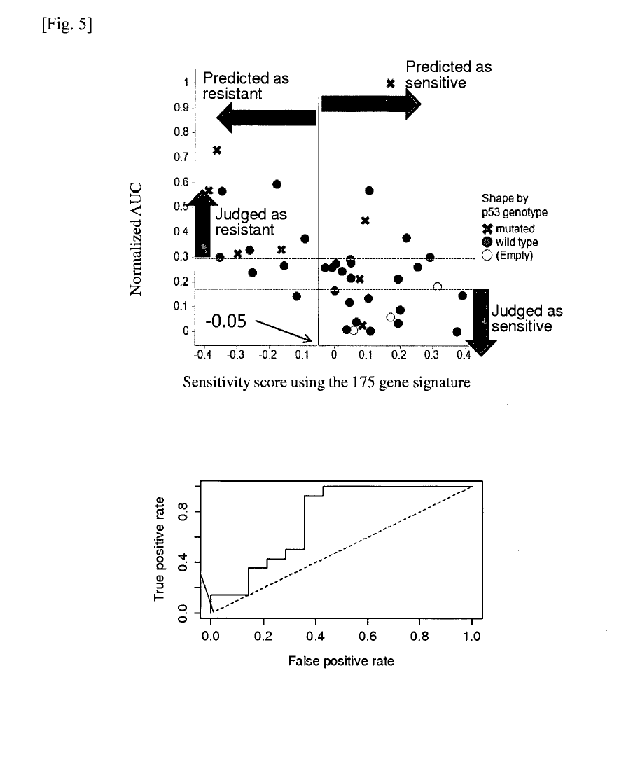

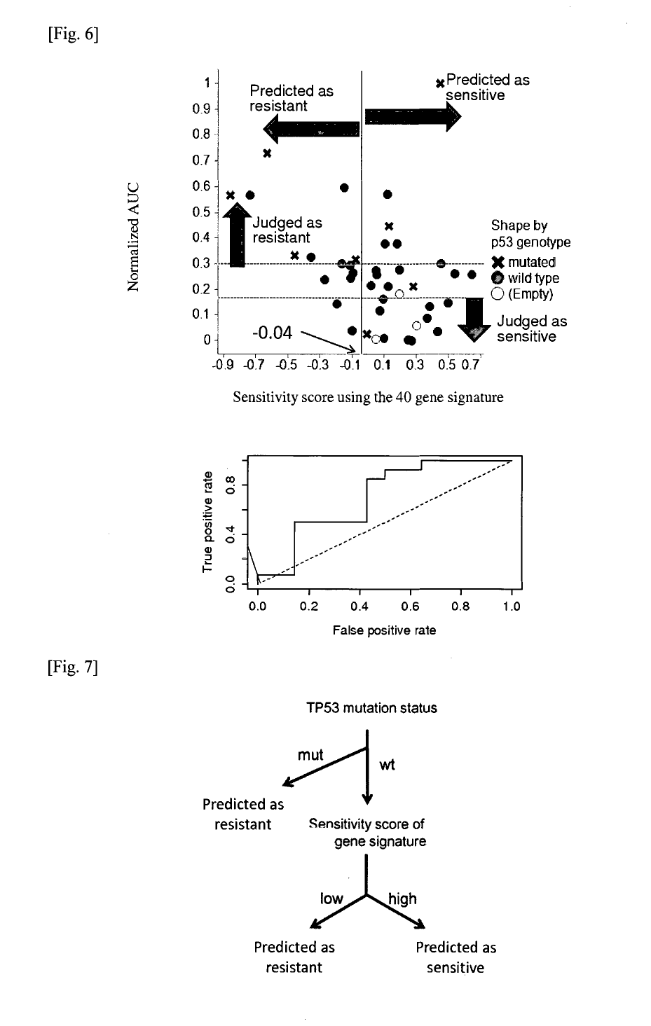

In the genotype mixed samples, 14 each sensitive and resistant samples were selected, and the 175-gene signature or 40-gene signature was applied. The prediction accuracy of the 175-gene signature was 79% (see FIG. 5 and Table 6), when the cutoff value was −0.05. Also, the prediction accuracy of the 40-gene signature was 71%, (see FIG. 6 and Table 7), when the cutoff value was −0.04.

TABLE 6
Prediction performance of sensitivity (all samples)

| | Number of the samples | Number of the samples |
|---|---|---|
| | predicted as resistant | predicted as sensitive |
| Number of the samples | 9 | 5 |
| which are resistant | | |
| Number of the samples | 1 | 13 |
| which are sensitive | | |

The prediction accuracy: (9 + 13)/28 × 100% = 79%

TABLE 7
Prediction performance of sensitivity (all samples)

| | Number of the samples | Number of the samples |
|---|---|---|
| | predicted as resistant | predicted as sensitive |
| Number of the samples | 8 | 6 |
| which are resistant | | |
| Number of the samples | 2 | 12 |
| which are sensitive | | |

The prediction accuracy: (8 + 12)/28 × 100% = 71%

These results suggest that the sensitivity to the test compound is predictable in TP53 wild type AML subjects, and in all AML subjects without determination of TP53 genotype. Further, it was noted that, in the TP53 mutant samples, the three sensitive samples were all predicted as sensitive and the other three resistant samples were predicted as resistant. Therefore, the sensitivity to the test compound is also predictable in TP53 mutant AML subjects.

We further showed the predictive performance when the 175-gene signature is applied after TP53 mutation status was incorporated as first predictive factor for resistance. Specifically, we predicted as resistant when the samples have TP53 mutations, and the predicted sensitivities to MDM2i of the rest of samples with wild-type TP53 were low using the gene signature and predicted as sensitive when the predicted sensitivities to MDM2i of the rest of samples with wild-type TP53 were high (see the general prediction scheme shown in FIG. 7). The prediction accuracy of the 175-gene signature was 82% (see Table 8), when the cutoff value was −0.5, showing highly accurate performance compared to TP53 mutation status alone or the gene signature alone.

TABLE 8
Prediction performance of sensitivity by the method of FIG. 7 (all
samples)

| | Number of the samples | Number of the samples |
|---|---|---|
| | predicted as resistant | predicted as sensitive |
| Number of the samples | 11 | 3 |
| which are resistant | | |
| Number of the samples | 2 | 12 |
| which are sensitive | | |

The prediction accuracy: (11 + 12)/28 × 100% = 82%

1. A pharmaceutical composition for use in treating acute myeloid leukemia (AML) in a patient in need thereof, comprising an effective amount of (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide or a salt thereof and a pharmaceutically acceptable carrier.

2. The pharmaceutical composition according to claim 1, wherein the salt is mono p-toluenesulfonic acid salt monohydrate.

3. The pharmaceutical composition according to claim 1, wherein the AML has been predicted as being sensitive to the treatment by measuring the expression level of at least one gene or all genes selected from a group of the 177 genes listed in FIG. 1 in a sample obtained from the patient.

4. The pharmaceutical composition according to claim 3, wherein the patient has been predicted as being sensitive to the treatment by measuring the expression levels of the 177 genes listed in FIG. 1 in a sample obtained from the patient.

5. The pharmaceutical composition according to claim 3, wherein the patient has been predicted as being sensitive to the treatment by measuring the expression levels of the 175 genes which are listed in FIG. 1 except for EDA2R and SPATA18 in a sample obtained from the patient.

6. The pharmaceutical composition according to claim 3, wherein the patient has been predicted as being sensitive to the treatment by measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in a sample obtained from the patient.

7. The pharmaceutical composition according to claim 3, wherein the patient has been predicted as being sensitive to the treatment by measuring the expression levels of at least one gene or all genes selected from the group of the genes below: RPS27L, FDXR, CDKN1A and AEN in a sample obtained from the patient.

8. The pharmaceutical composition according to claim 3, wherein the patient has been predicted as being sensitive to the treatment by measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in a sample obtained from the patient.

9. The pharmaceutical composition according to claim 3, wherein the patient has been predicted as being sensitive to the treatment by measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in a sample obtained from the patient.

10. The pharmaceutical composition according to claim 3, wherein the patient has a wild type TP53 gene in the genome of AML cells to be treated.

11. A method of treating acute myeloid leukemia (AML) in a patient in need thereof, comprising administering to the patient an effective amount of (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide or a salt thereof.

12. The method according to claim 11, wherein the salt is mono p-toluenesulfonic acid salt monohydrate.

13. The method according to claim 11, wherein the patient has been predicted as being sensitive to the treatment by measuring the expression level of at least one gene or all genes selected from a group of the 177 genes listed in FIG. 1 in a sample obtained from the patient.

14. The method according to claim 13, wherein the patient has been predicted as being sensitive to the treatment by measuring the expression levels of the 177 genes listed in FIG. 1 in a sample obtained from the patient.

15. The method according to claim 13, wherein the patient has been predicted as being sensitive to the treatment by measuring the expression levels of the 175 genes which are listed in FIG. 1 except for EDA2R and SPATA18 in a sample obtained from the patient.

16. The method according to claim 13, wherein the patient has been predicted as being sensitive to the treatment by measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in a sample obtained from the patient.

17. The method according to claim 13, wherein the patient has been predicted as being sensitive to the treatment by measuring the expression levels of at least one gene or all genes selected from the group of the genes below: RPS27L, FDXR, CDKN1A and AEN in a sample obtained from the patient.

18. The method according to claim 13, wherein the patient has been predicted as being sensitive to the treatment by measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in a sample obtained from the patient.

19. The method according to claim 13, wherein the patient has been predicted as being sensitive to the treatment by measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in a sample obtained from the patient.

20. The method according to claim 13, wherein the patient has a wild type TP53 gene in the genome of AML cells to be treated.

21. A method of predicting sensitivity to MDM2i treatment of AML in a patient suffering from AML, comprising measuring the expression levels of at least one, at least two, at least three, at least four or all of the 177 signature genes shown in FIG. 1, wherein the MDM2i is (3′R,4′S,5′R)—N-[(3R,6S)-6-Carbamoyltetrahydro-2H-pyran-3-yl]-6″-chloro-4′-(2-chloro-3-fluoropyridin-4-yl)-4,4-dimethyl-2″-oxo-1″,2″-dihydrodispiro[cyclohexane-1,2′-pyrrolidine-3′,3″-indole]-5′-carboxamide or a salt thereof.

22. The method according to claim 21, comprising measuring the expression levels of at least one, at least two, at least three, at least four or all of the 175 signature genes which are the genes presented in FIG. 1 except for EDA2R and SPATA18.

23. The method according to claim 21, comprising measuring the expression levels of at least one, at least two, at least three, at least four or all of the forty signature genes consisting of BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC.

24. The method according to claim 21, comprising measuring the expression levels of RPS27L, FDXR, CDKN1A and AEN.

25. The method according to claim 21, further comprising determining whether or not the AML has wild-type TP53 gene in its genome.

26. A method of predicting sensitivity to MDM2i treatment in a patient suffering from AML comprising,
determining whether or not the AML has mutant TP53 gene in its genome,
when the AML has mutant TP53 gene, then the patient is predicted as resistant, and
when the AML has wild-type TP53 gene, subsequently measuring the expression levels of at least one, at least two, at least three, at least four or all of the 177 signature genes shown in FIG. 1 in the AML,
when the AML has a low signature score compared to a predetermined cutoff value, then the patient is predicted as resistant, and
when the AML has a high signature score compared to the predetermined cutoff value, then the patient is predicted as sensitive.

27. The method according to claim 26, wherein the step of measuring is measuring the expression levels of at least one gene or all genes selected from the group of the genes below: BAX, C1QBP, FDXR, GAMT, RPS27L, SLC25A11, TP53, TRIAP1, ZMAT3, AEN, C12orf5, GRSF1, EIF2D, MPDU1, STX8, TSFM, DISC1, SPCS1, PRPF8, RCBTB1, SPAG7, TIMM22, TNFRSF10B, ACADSB, DDB2, FAS, GDF15, GREB1, PDE12, POLH, C19orf60, HHAT, ISCU, MDM2, MED31, METRN, PHLDA3, CDKN1A, SESN1 and XPC in the AML.

28. The method according to claim 26, wherein the signature genes is at least one gene or all genes selected from the group of the genes below: RPS27L, FDXR, CDKN1A and AEN in the AML.

29. The method according to claim 26, wherein the step of measuring is measuring the expression levels of at least one gene or all genes selected from the group of the genes below: BAX, RPS27L, EDA2R, XPC, DDB2, FDXR, MDM2, CDKN1A, TRIAP1, BBC3, CCNG1, TNFRSF10B, and/or CDKN2A in the AML.

30. The method according to claim 26, wherein the step of measuring is measuring the expression levels of at least one gene or all genes selected from the group of the genes below: BAX, RPS27L, XPC, DDB2, FDXR, MDM2, CDKN1A, AEN, RRM2B, SESN1, CCNG1, ZMAT3, and/or TNFRSF10B in the AML.
